(19) United States
(12) Patent Application Publication
(10) Pub. No.: US 2008/0300691 A1
(43) Pub. Date: Dec. 4, 2008

(54) Biomimetic Synthetic Nerve Implant Casting Device

(75) Inventors: Mario I. Romero-Ortega, Coppell, TX (US); Pedro Galvan-Garcia, Irving, TX (US)

Correspondence Address: VINSON & ELKINS, L.L.P., FIRST CITY TOWER, 1001 FANNIN STREET, SUITE 2500, HOUSTON, TX, 77002-6760, US

(73) Assignee: Texas Scottish Rite Hospital for Children, Dallas, TX (US)

(21) Appl. No.: 11/939,483

(22) Filed: Nov. 13, 2007

Related U.S. Application Data
(62) Division of application No. 11/418,927, filed on May 5, 2006.
Application No. 11/418,927 is a continuation-in-part of application No. PCT/US04/38087, filed on Nov. 5, 2004.
(60) Provisional application No. 60/517,572, filed on Nov. 5, 2003.

(51) Int. Cl. A61F 2/02

(52) U.S. Cl. 623/23.72

(57) ABSTRACT

A biomimetic biosynthetic nerve implant (BNI) casting device includes a matrix casting tube; a matrix casting tube protective shield comprising a male coupling portion joinable to a female coupling portion, wherein the joined portions encase the matrix casting tube; microchannel forming fibers; a fixing point for holding one end of the microchannel forming fibers; loading fiber guideholes for placement of the microchannel forming fibers; one or more ports for injection of matrix material into the casting tube; and a cell suspension loading well in fluid communication with the matrix casting tube when the device is fully assembled such that removing the fibers from the formed implant can draw fluid containing cells and/or other agents into the microchannels.

### CROSS-REFERENCES TO RELATED APPLICATIONS

The present application is divisional application of copending U.S. Ser. No. 11/418,927, filed May 5, 2006, which is a continuation-in-part of PCT/US04/38087, filed Nov. 5, 2004, designating the United States of America and published in English, which claims the benefit of U.S. Provisional Application No. 60/517,572, filed Nov. 5, 2003. Each of the above-identified applications is hereby incorporated by reference in its entirety for all purposes.

### STATEMENT REGARDING FEDERALLY SPONSORED RESEARCH OR DEVELOPMENT

Not applicable.

### REFERENCE TO A “Microfiche Appendix”

Not applicable.

### BACKGROUND OF THE INVENTION

1. Field of the Invention

The present disclosure relates to biomimetic biosynthetic nerve implants for nerve repair, for example spinal cord injury repair.

2. Description of Related Art

Injuries to the adult nervous system are irreversible and bear long lasting functional deficits. The total costs for the first year of care of paraplegic and quadriplegic patients has been estimated at $152,000 and $417,000 respectively, and the lifetime care of a 25-year-old paraplegic patient is about $750,000 (www.neurolaw.com). Although numerous approaches have been proposed to repair the injured central (brain and spinal cord) and peripheral (sensory ganglia and sensori-motor nerves) nervous system, repair strategies that require tissue implantation for bridge repairs have not matured yet into clinical practice.

Several hundred thousand peripheral nerve injuries occur each year in Europe and the United States, mainly as a result of trauma to the upper extremity. It is estimated that approximately 200,000 nerve repair procedures are performed annually in the U.S. alone. (Archibald et al., J. Comp. Neurol 306, 685-96, 1991; Evans, Anat. Rec 263, 396-404). Nerve gaps from segmental tissue loss are routinely repaired by transplanting autogenous nerve grafts; however, this currently accepted “gold-standard” technique results in disappointingly poor (0-67%) functional recovery at the expense of normal donor nerves. (Allan, C. H. Hand Clin 16, 67-72, 2000; Kline et al., J Neurosurg 89, 13-23, 1998). The first use of nerve grafts in humans was reported in 1878, but the wide use of this technique was developed during World War II when nerve grafting became the standard method for nerve-gap repair. Harvesting of nerve grafts results in co-morbidity that includes scarring, loss of sensation, and possible formation of painful neuroma. The donor nerves often are of small caliber and limited number. As functional recovery in peripheral nerve reconstruction is poor, clearly, an alternative method for bridging nerve gaps is needed. (Dellon et al., Plast Reconstr Surg 82, 849-56, 1988).

Tissue engineering aims at making virtually every human tissue. Potential tissue-engineered products include cartilage, bone, heart valves, muscle, bladder, liver, and nerve. For nerve gap repair, tabularization techniques have been extensively studied as a possible method to bridge the gap. Substantial nerve regeneration, however, has never been reported in the reconstruction of human major nerves using silicone tubing. (Braga-Silva, J Hand Surg [Br] 24, 703-6 1999; Lundborg, et al., J Hand Surg [Br] 29, 100-7, 2004). Despite the fact that the peripheral nerve has an excellent capability of regenerating after a lesion, the main problem is its lack of superior functional recovery compared to autologous nerve repair. A factor contributing to this limitation is perhaps the lack of specificity at the time of reinervating original targets (Alzate et al., Neurosci Lett, 286, 17-20, 2000). To improve on directed target reinervation and functional recovery, biodegradable synthetic conduits have not only included biodegradable nerve guides (Kiyotani, T. et al. Brain Res 740, 66-74, 1996; Rodriguez et al., Biomaterials 20, 1489-500, 1999; Weber et al., Plast Reconstr Surg 106, 1036-45; discussion 1046-8, 2000), but also the incorporation of exogenous factors such as extracellular matrix molecules (Yoshii et al., J Biomed Mater Res 56, 400-5 2001), cell adhesion molecules (Matsumoto, K. et al. Brain Res 868, 315-28, 2000), growth factors (Ahmed, et al., Z Scand J Plast Reconstr Surg Hand Surg 33, 393-401, 1999; Fine, Eur J Neurosci 15, 589-601, 2002; Midha et al., J Neurosurg 99, 555-65, 2003; Rosner et al., Ann Biomed Eng 31, 1383-401, 2003; Lee, A. C. et al. Exp Neurol 184, 295-303, 2003), or cells such as Schwann or bone marrow stromal stem cells (Ansselin, et al., Neuropathol Appl Neurobiol 23, 387-98, 1997; Frostick et al., Microsurgery 18, 397-405, 1998; Dezawa et al., Eur J Neurosci 14, 1771-6, 2001). However, only modest results of nerve regeneration and functional recovery have been reported (Gordon et al., J Peripher Nerv Syst 8, 236-50, 2003; Schmidt et al., Annu Rev Biomed Eng 5, 293-347, 2003).

Optimally, tabularization repair designs should approximate closely the cytoarchitecture of the native peripheral nerve, as well as provide proper cellular and molecular cues to entice and direct axonal regeneration. Attempts to mimic the nerve tissue by other investigators have used longitudinally oriented bioabsorbable filaments to direct axonal growth (Ngo et al., J Neurosci Res, 72, 227-238, 2003), and PGA collagen tubes filled with laminin-coated collagen fibers (Yoshii et al., J Biomed Mater Res, 56, 400-405, 2001). A tubular nerve guidance conduit possessing the macroarchitecture of a polyfascicular peripheral nerve has been reported (U.S. Pat. Nos. 6,214,021, 6,716,225). However, there are several limitations. The manufacture of nerve conduit is rather complicated, it is time consuming, and in most cases requires the use of solvents toxic to the cells. The dynamic seeding of Schwann cells requires special equipment, involves multiple steps, and the procedure for loading of cells alone can take several hours. In addition, the material for the conduit is not transparent, and thus not suitable for real time observation and dynamic follow up of cellular and/or tissue morphology and viability. Thus, despite the recent progress in the engineering of biosynthetic nerve prosthesis, no current design closely resembles the natural morphology of multiple fascicular compartments in the peripheral nerve.

To better resemble the natural microanatomy of peripheral nerves, novel polymer scaffolds are specifically designed to form organized arrays of open microtubules (Hadlock et al., Tissue Eng., 2000, 119-127). One drawback of current methods of multiluminal nerve repair is that they require rather complicated fabrication techniques. Quite often the evidence for the functional efficacy of such techniques is either incomplete or entirely absent (Hadlock et al., Tissue Eng., 2000; Moore et al., Biomaterials, 2006, 419-429; Stokols and Tuszynski, Biomaterials, 2006, 443-451). We developed a simple and reproducible method for the fabrication of biosynthetic nerve implants that provides multiple and physically permissive contact guidance structures (agarose microchannels), each loaded with favorable biological substrates (ie., collagen/cells) for nerve growth.

The lack of endoneural tube-like structures in several types of nerve grafts have proven to be an impediment for proper nerve regeneration (Fansa et al., Neurol Res 26, 167-73, 2004). To address this problem, an agarose-based multi-channel matrix has been developed, that allows for the controlled culture and evaluation of cellular elements, both normal or genetically-engineered, and seeded into longitudinally arranged channels (US Application Publication No. 20030049839). This idea has been supported by others, who have reported multiple microchannel matrices made by embedding extruded polycaprolactone fibers into poly 2-hydroxyethyl methacrylate (pHEMA) hydrogels and then dissolving the fibers in acetone (Flynn et al., Biomaterials 24, 4265-72, 2003), or by freeze-drying processing in agarose (Stokols et al., Biomaterials 25, 5839-46, 2004). Several problems still limit the effectiveness of organ bioengineering, and in particular the production of a biomimetic implant. For example, some hydrogels like pHEMA and agarose are inert and cells do not attach to them, requiring the modification of these polymers with permissive peptide derivatives (Yu et al., Tissue Eng 5, 291-304, 1999; Luo et al., Nat Mater 3, 249-53, 2004). Additionally, cellular growth within the microchannels occurs in the luminal space only with the addition of extracellular matrix molecules (ECM). Unfortunately, the variable availability and degradation of ECM limits cellular growth within the microchannels and thus, their capacity to provide a uniform cellular scaffold for cell growth. There is still a need, therefore for a tissue engineering scaffold that serves as a three-dimensional (3-D) template for initial cell attachment and subsequent tissue formation both in vitro and in vivo, that provides the necessary support for cells to attach, proliferate, and maintain their differentiated function, and that can provide the physical and biochemical support upon which the cellular components can be positioned in order that they may develop and achieve optimal organ growth, and especially for nerve growth.

Biodegradable polymers have been used in the surgical repair of peripheral nerves, but their potential for use in the central nervous system has not been exploited adequately. The use of a biodegradable polymer implant has the dual advantages of providing a structural scaffold for axon growth and a conduit for sustained-release delivery of therapeutic agents. As a scaffold, the microarchitecture of the implant can be engineered for optimal axon growth and transplantation of permissive cell types. As a conduit for the delivery of therapeutic agents that may promote axon regeneration, the biodegradable polymer offers an elegant solution to the problems of local delivery and controlled release over time. Thus, a biodegradable polymer graft would theoretically provide an optimal structural, cellular, and molecular framework for the regrowth of axons across a spinal cord lesion and, ultimately, neurological recovery. (Friedman et al., Neurosurgery, 2002, discussion 751-742). The complex nature of spinal cord injury appears to demand a multifactorial repair strategy. One of the components that will likely be included is an implant that will fill the area of lost nervous tissue and provide a growth substrate for injured axons. (Oudega et al., Braz. J. Med. Biol. Res., 2005, 825-835) The histopathological reaction of the mammalian lesioned spinal cord, when adequately directed by a scaffolding structure can be beneficial for the expression of the intrinsic regenerative capacity of the spinal cord tissue. (Marchand and Woerly, 1990, Neuroscience, 1990, 45-60)

### BRIEF SUMMARY OF THE INVENTION

The present disclosure may be described in certain aspects as novel designs for a biosynthetic nerve implant (BNI), which incorporate state of the art biomaterial technology and provide enhanced and directed nerve regeneration both in the peripheral nervous system as well as in the adult injured spinal cord, as compared to other techniques. Advances provided in the disclosure include design of the implant amenable to nanotechnology incorporation, design of a novel scaffold-casting device for medical-grade production, and definition of the cellular and molecular components. The present disclosure includes initial animal evidence demonstrating at the anatomical, behavioral, and electrophysiological levels, that the disclosed BNI better promotes and directs nerve regeneration after sciatic nerve gap repair and dorsal hemisection gap repair of the adult spinal cord.

Preferred embodiments of the disclosure include a biosynthetic nerve scaffold that provides an external, perforated conduit incorporating multiple microchannels within the lumen and including a biodegradable hydrogel matrix. Furthermore, each microchannel may incorporate cells, growth factors and/or extracellular matrix molecules both in the lumen and/or in the walls of the microchannel (FIG. 1). In preferred embodiments, micro- or nanostructures are incorporated in the lumen and/or luminal surface of the microchannels. In some embodiments, a gel-forming matrix is used with the cells in the lumen. When, in certain preferred embodiments, cultured Schwann cells (SCs) are loaded into these channels, the cells are attached to the surface of the microchannels by virtue of a molecularly defined lumen that permits cells to elongate into a three-dimensional viable tissue structure within hours. The early presence and interaction of extracellular matrices components, either natural or synthetic, and/or cellular components, either natural or genetically modified, and the novel incorporation of multiple luminal microdomains within the microchannels, designed for molecular, pharmacological, or electrophysiological manipulations or readings, provide an ideal environment for stimulation and study of the early phases of axon regeneration.

By forming a permissive substrate for selective neural growth, the initial nerve regeneration events occur faster, and regeneration is accelerated. Although not wishing to be limited to any theory, providing microspheres within the microchannels is contemplated as allowing for the Schwann cells/hydrogel mixture to anchor to the luminal surface of the microchannels. The formed Schwann cell cable is then continuous and somewhat uniform along the microchannels, which is an intuitively better biosynthetic conduit for nerve repair, with a higher potential of improving functional recovery. The present disclosure is not limited to regeneration of nerve cell connections or to nerve tissue of either the central or peripheral nervous systems. The transparent nature of the hydrogel used for casting the nerve scaffold allows for real time observation and dynamic follow up of cellular viability and morphology prior to implantation. Therefore, this disclosure further provides novel methods and compositions for testing the effect(s) of biologically active agents on various cell types.

The present disclosure also provides a specially designed, three-dimensional scaffold-casting device that is particularly suited for making the tissue scaffolds in a reproducible and sterile manner. The device may function to fabricate a multi-luminal implant scaffold matrix to selectively present molecules or seed cells spatially and temporally in three-dimensions with the required physical, structural, biological and chemical factors to promote cellular development. The disclosed devices are suitable for the production and reproduction of bio-engineered 3-D cellular scaffolds to exact specifications and requirements for basic research and clinical applications in tissue bioengineering, allowing for the effective reproduction and repair of various specialized tissue types and organs by directly addressing the highly complex, three-dimensional, cellular architectural morphology.

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a schematic drawing of a model of a biosynthetic nerve implant (BNI). The hydrogel-based multi-luminal scaffold is designed to allow fascicular growth of axons through the multiple microchannels. The main components are an external perforated conduit, pertinent for peripheral nerve gap repair but not spinal cord injury repair, and an internal multi-luminal matrix. Each microchannel of the multi-luminal matrix may incorporate cells or molecules in the lumen, and/or micro-structures or nano-domains either in the lumen or embedded in the walls of the microchannels, in order to present extracellular matrix molecules and growth factors to the regenerating nerves. Furthermore, these domains, molecules and/or cells inside each microchannel, can be used to evaluate and quantify cellular growth and function. The hydrogel-based multi-luminal scaffold is designed to allow compartmentalization of the regenerated nerve tissue and segregation and directed growth through the combination of physical microchannels and specific molecular cues.

FIG. 2 is a schematic view of an external perforated conduit. A perforated conduit, for example, either non-bioreabsorbable polyurethane tubes or tubes made of biodegradable material such as collagen, PLA, caprolactone, or others, are designed not only to provide continuity of the transected nerve ends, but also for protection and facilitation of nutrients and gas exchange for the cells seeded within the multi-luminal channels. The exemplary device in FIG. 2 serves as a three-dimensional multi-luminal nerve implant matrix casting tube. FIG. 2A is an oblique view of a tube showing the external wall of the tube including the conical holes, and the internal lumen of the tube. In certain preferred embodiments, the conical holes are spaced 2 mm apart and the internal lumen is preferably 1.68 mm in diameter. FIG. 2B is a longitudinal sectional view of the wall of a tube going through the central axis of the conical holes. FIG. 2C shows a transverse sectional view of the tube, and the placement of the conical holes. In certain preferred embodiments the conical holes have an external diameter of 0.25 mm and an internal diameter of 0.1 mm.

FIG. 3 consists of two photographs of a hand-made prototype of a BNI-casting device. Panel A is a device made of dental cement (a), with plastic fibers (b) guided through it by a series of holes cast at both ends of the device. The device has a matrix casting well (c) to accommodate the external tubing, and a loading well (d) for the placement of cell suspensions and/or molecules that can then be loaded into the hydrogel matrix simply by removing the plastic fibers once the hydrogel has polymerized. Panel B shows the detail of the internal design of the casting device and indicates the area for the coupling of the external tubing, as well as the aligned fibers in place. Scale bar=0.5 cm.

FIG. 4 shows nerve repaired using a Multiluminal peripheral nerve repair through the BNI. Adult rat sciatic nerve are shown at 10 weeks post gap repair either by autograft (A), collagen-filled tubularization (B), collagen-loaded 7-channel BNI with external tubing (C, E, G) and 14-channel BNI without the external tubing (D, F, H). Multiple nerve cables regenerated in the BNI-repaired animals through the available microchannels. Vascularization is indicated in both the intraluminal nerve cables (arrowheads) as well as in the outer mesenchimal membrane (arrows). Scale bars=2 mm (A), 400 μm (E). The plurality of openings extending radially through the PTFE tubing facilitated cell migration and vascularization in both repair methods. In the BNI, however, cells migrated into the space between the external tubing and the multi-luminal matrix so that a highly vascular cellular capsule is formed and nutrient and gas exchange with the intra-luminal cellular structures is favored (arrows in H).

FIG. 5 shows fascicular-like repair in the 14-channel BNI. Toludine blue-stained sections of uninjured controls (A), autograft (B), collagen-filled tube (C), and BNI repaired (D, E) sciatic nerves. A mesenchimal layer was observed to cover the outer surface of the BNI hydrogels (arrow). Higher magnification of the insert in D, shown in (E), shows a perineurium-like layer (arrows), blood vessels (arrowheads), and densely packed axons regenerating within the BNI microchannels. Quantification of the total area of nerve regeneration indicates that BNI repair offered limited available area for nerve repair compared to the other methods (F). P≦0.01 vs normal and P≦0.001 vs autograft. Scale bars=400 μm (A), 50 μm (E).

FIG. 6 Shows increased axon regeneration density in multiluminal repair. Electron microscopy photographs of uninjured normal controls (A), and those repaired by autograft (B), collagen-loaded tube (C), and 14-channel BNI (D), after sciatic nerve transection. Quantification of myelinated (E) and unmyelinated (F) axons within a 0.033 mm² area revealed a reduced number of both axon types in uninjured animals compared to those with tube/collagen, autograft, and BNI treatments. Separate analysis of axon diameter distribution (G), and myelin thickness (H), shows increased numbers of 4-6 μm axons in both the autograft and the BNI groups, and reduced myelination compared to the normal animals. *=P≦0.05; **=P≦0.01. Scale bar=10 μm.

FIG. 7 shows sensory-motor neuron regeneration. Regenerated motorneurons in the ventral spinal cord (VMN; A-C) and sensory neurons in the dorsal root ganglia (DRG; D-F), were visualized with Nissl staining and identified by FluoroGold (FG) tracing of their regenerated axons distal to the grafted implant (G). FG+ VMN (H) and DRG (I) cells were quantified in all groups. *=P≦0.01 Scale bar=50 μm.

FIG. 8 shows functional recovery mediated by repaired peripheral nerves. Behavioral response to plantar sensory stimulation in autograft (A), tube/collagen (B) and BNI (C), showed gradual improvement in all groups. A similar trend was observed after motor function was evaluated using the digit abduction scoring assay (D). Electrophysiological testing (E) demonstrated electrical conduction and myelectric depolarization in both the tube/collagen and the BNI groups.

FIG. 9 demonstrates the use of the BNI implant in repairing the injured spinal cord. (A) Schematic representation of a coronal view of the spinal cord after dorsal hemisection and placement of the BNI which contains Schwann cell in the channels. (B) Photograph of Schwann cells cultured in the BNI 24 hrs in culture and prior to implantation. (C) Photograph of the injured spinal cord 45 days after repair. Regenerated tissue is evident inside the microchannels (arrows). (D-E) Histological staining of the repaired spinal cord in a longitudinal (D) and coronal (E) section showing successful tissue regeneration though the BNI microchannels.

FIG. 10 Photograph of the injured spinal cord 45 days after repair. Regenerated tissue is evident inside the microchannels (arrows). Numerous cells are located inside each microchannel as indicated by the nuclear staining DAPI. The implanted GFP-labeled Schwann cells survived inside the microchannels as indicated in the GFP and Merged photographic panels.

FIG. 11 shows a higher magnification of the regenerated tissue inside a BNI microchannel in the injured spinal cord, 45 days after repair. Numerous cells are visualized inside the microchannel as indicated by the nuclear staining DAPI. The implanted GFP-labeled Schwann cells survived inside the microchannels as indicated in the GFP (arrows) and numerous regenerated axons, visualized with the specific neuronal marker b-tubulin (arrow heads), demonstrated successful guided nerve regeneration in the injured adult spinal cord.

FIG. 12 shows several designs of the BNI. Additional modification of the guiding ports for fiber placement are shown to achieve different microchannel sizes or shapes (Panels A-C). Modifications can also be included to either preserve the physical isolation of the regenerated tissue inside the BNI (A), or to allow a connection between the outside tissue and specific microchannels (B-D). In such cases a single or a plurality of external pins can be placed through the perforations of the external tubing prior to hydrogel polymerization. Subsequent removal of these pins then produces interconnected channels within the BNI (arrows). The ability of combining different microchannel size, shape, and interconnectivity, results in several combinatorial designs that not only provide better tissue regeneration capacity but also entice the growth of endogenous cells into the multi-luminal matrix of the BNI, thus increasing the potential for vascularization and improved functional outcomes. In doing so, it offers the alternative of directing endogenous cells into the BNI for tissue regeneration in certain embodiments, rather than incorporating exogenous cells into the implants.

FIG. 13 shows longitudinal and cross sectional views of a casting device. The left panel is a graphic representation of a partially disassembled BNI three-dimensional nerve implant casting device viewed through a horizontal plane of section. This figure also shows a graphic representation of a transverse sectional view, indicated by arrows at levels (A-F) of the BNI three-dimensional nerve implant casting device. The plane of section through (A) shows the matrix injection coupling port (b-c), and the body of the matrix injection coupler (a). Shown in (B) is one of the guide holes for the conduit-casting/cell-suspension loading fibers indicated by (e) and one of the matrix injection ports indicated by (d). The section through (C) shows the male coupling portion of the protective shield of the matrix casting-tube (f). (D) Shows the matrix casting implant tube (g). The suspension loading well (h) is seen in the cross-section through the body of the distal end of the BNI implant casting device (E). The section through (F) shows the wall of the projection for the inert, non-reactive, rubber plug (1) for cell suspension injection and the matrix overflow ports indicated by (k).

FIG. 14 is a graphic representation of a fully assembled three-dimensional nerve implant casting device showing a view through a central sagittal plane of section (A), which also shows the internal cell-suspension loading well air bleeder port (a) and a view through a central horizontal plane of section (B) as shown in FIG. 13.

FIG. 15 is a graphic representation of a conduit-casting/cell-suspension loading fiber for modification with molecular micro-domains of the luminal surface of a multi-conduit cellular scaffold. An oblique view of the fiber (A) shows the solid fiber (a); with a coating (b), for anchoring and subsequent release of the carrier micro- or nano-particles or packets (c). The different components of the assembly are shown in a cross-sectional view in (B).

FIG. 16 shows microphotographs demonstrating the process of incorporation of 10 μm latex beads into the luminal surface of the microchannels. (A) Shows a plastic fiber coated with latex beads and used to cast agarose matrix microchannels. (B) Illustrates the microchannel cast after removal of the plastic fiber, leaving behind the micro-beads embedded into the agarose and, thus, incorporated into the luminal surface of the channel. The lumen of this particular channel is empty, to demonstrate that the beads are indeed attached to the matrix. A higher magnification photograph of the microchannel shows in detail the embedded micro-beads (C, D). A transverse cryosection through a microchannel shows clear incorporation of the beads onto the luminal surface of the channel. Longitudinal and horizontal sections confirm this finding and are illustrated in (C) and (D), respectively.

### DETAILED DESCRIPTION OF THE INVENTION

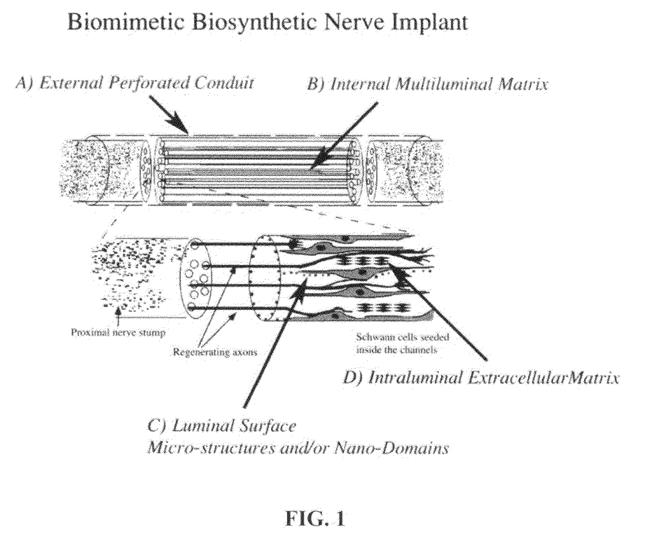

An embodiment of the disclosure is shown in FIG. 1, and provides a casting device useable to cast a multi-luminal scaffold a novel biosynthetic nerve implant. The device includes an outer biodegradable or non-biodegradable tube or sleeve with a plurality of perforations to allow cellular migration inside the lumen, a multi-laminal matrix with multiple microchannels, which in turn can be loaded with single or multiple selected cell types or molecules. The surface area of each microchannel can be further modified to incorporate micro- or nano-domains that are cast into the microchannels during the extraction of the conduit-casting/cell suspension loading fibers. In certain embodiments, the fibers are coated with chemically treated, cell-anchoring, nano- or micro-structures, or a combination thereof. The micro- or nano-structures are released and remain embedded in the matrix upon extraction of the fibers, which also draws cells or molecules into the lumen of each microchannel. The preferred nerve conduit provides great flexibility for custom fabrication of a cell scaffold designed for a particular nerve to be repaired.

Preferred casting devices allow for the reproducible production of a nerve conduit with relative ease, and within a short period time. The hydrogel-based multi-luminal scaffold is designed to allow fascicular growth of axons through the multiple microchannels. As indicated in FIG. 1, each microchannel of the multi-luminal matrix may incorporate cells or molecules in the lumen, and/or micro-structures or nano-domains either in the lumen or embedded in the walls of the microchannels, in order to present extracellular matrix molecules and growth factors to the regenerating nerves. Furthermore, these domains, molecules, and/or cells inside each microchannel are used to evaluate and quantify cellular growth and function. The hydrogel-based multi-luminal scaffold is designed to allow compartmentalization of the regenerated nerve tissue and segregation and directed growth through the combination of physical microchannels and specific molecular cues.

The external conduit is preferably a tube composed of biocompatible and/or bioresorbable material(s). Such materials may include, but are not limited to cellulose, hydroxymethyl cellulose, hydroxyethyl cellulose, carboxymethyl cellulose, carboxymethyl chitosan, poly-2-hydroxyethyl-meth-acrylate, poly(R-3-hydroxybutyric acid-co-(R)-3-hydroxyvaleric acid)-diol (PHB), collagen, keratin, gelatin, glycinin, synthetic polymers, including polyesters such as polyhydroxyacids like polylactic acid (PLA), polyglycolic acid (PGA) and copolymers thereof such as poly(lactic acid-co-caprolactone), some polyamides and poly(meth)acrylates, polyanhdyrides, as well as non-degradable polymers such as polyurethane, polytrafluoroethylene, ethylenevinylacetate (EVA), polycarbonates, and some polyamides-methyl, or silicone rubber.

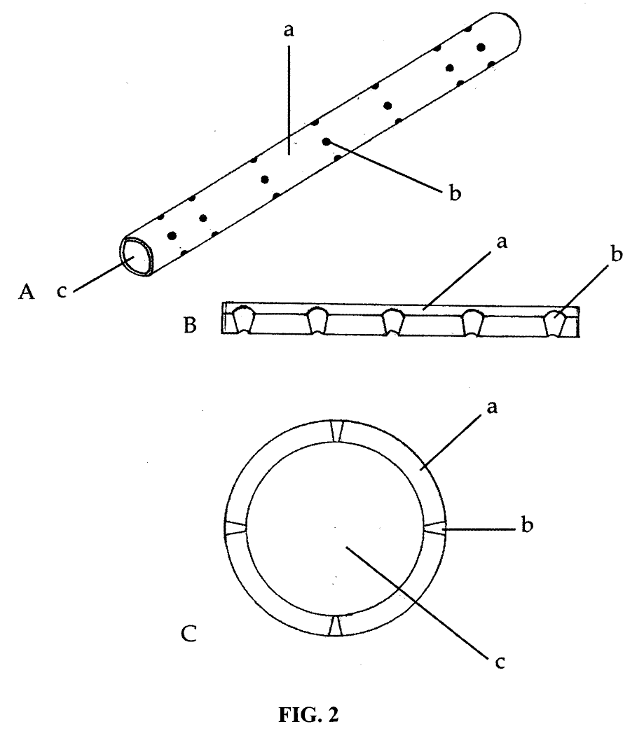

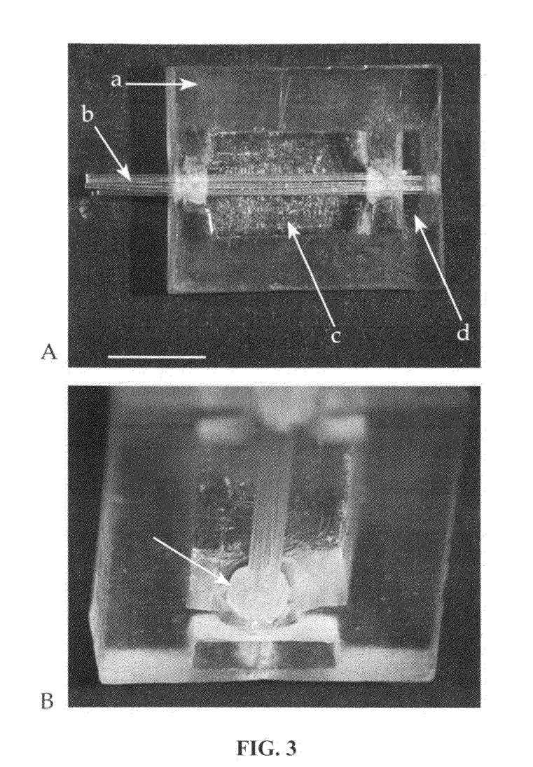

The perforations in the external conduit are designed to facilitate the migration of endogenous cells, such as those in the muscular fascia, which then vascularize the intra-luminal matrix, providing enhanced exchange of nutrients and gas for the cells seeded within the multi-luminal channels or the regenerated tissue. FIG. 2 shows a graphic representation of the three-dimensional multi-luminal nerve implant matrix casting tube.

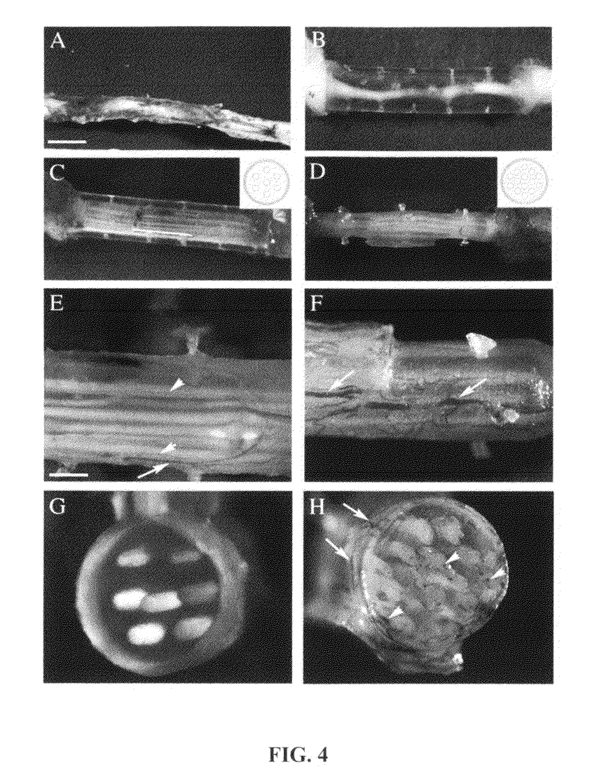

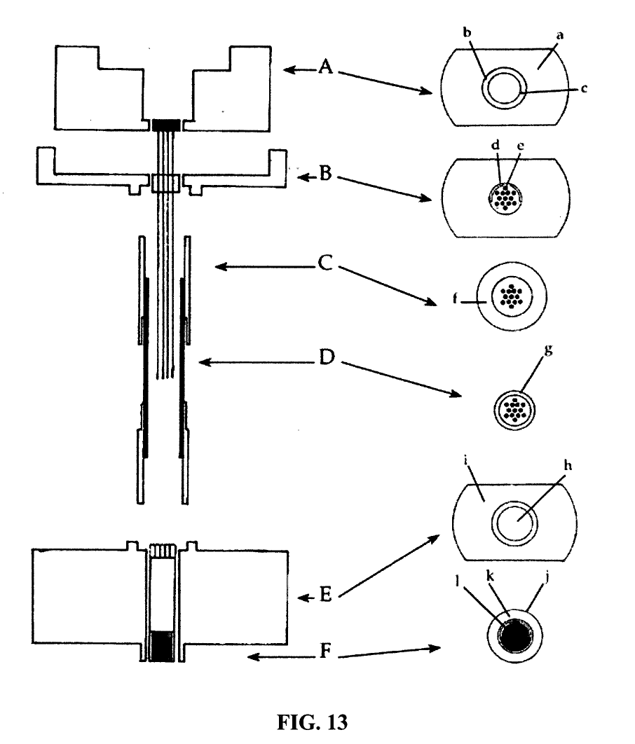

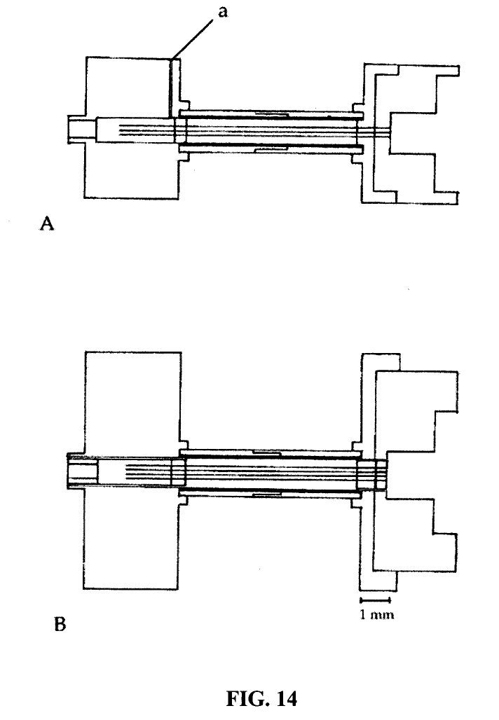

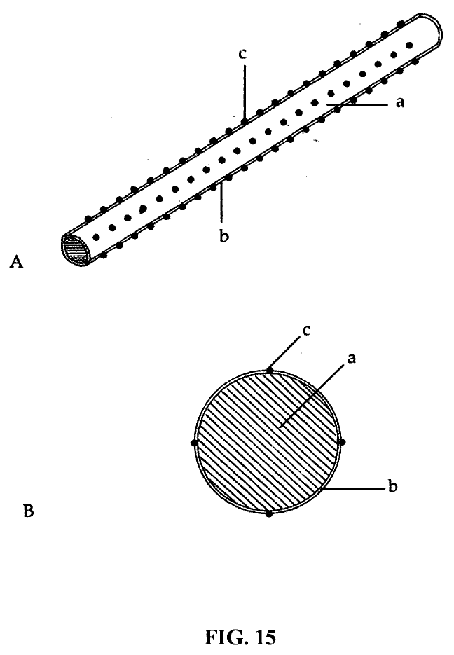

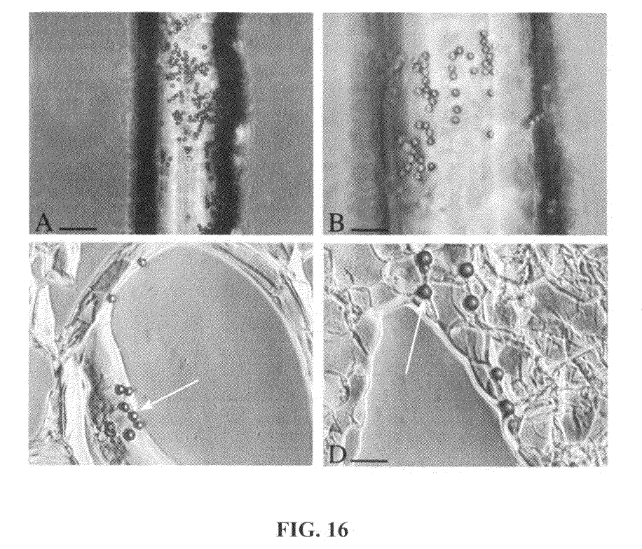

Hand-made prototypes of a BNI matrix-casting device were built (FIG. 3) to demonstrate the principle disclosed herein. The device, made of dental cement, has plastic fibers guided through it by a series of holes cast at both ends of the device. The device has a matrix casting well to accommodate the external tubing and a loading well for the placement of cells and/or molecules that are loaded into the hydrogel matrix simply by removing the plastic fibers once the hydrogel has polymerized. The microchannels may be geometrically distributed in different shapes and sizes to maximize tissue regeneration and to better match the fascicular nature of the specific nerve to be repaired. An advanced casting device is illustrated in FIGS. 13-15.

The multi-luminal matrix is made by casting multiple cylindrical microchannels within a biocompatible and bioresorbable, biopolymeric material capable of forming a hydrogel, wherein the cylindrical microchannels are formed inside the external tubing and parallel to the longitudinal axis of the tube; each cylindrical matrix has two ends. The intra-luminal matrix may include a material selected from the group consisting of agar, agarose, gellan gum, arabic gum, xanthan gum, carageenan, alginate salts, bentonite, ficoll, pluronic polyols, CARBOPOL, polyvinylpyrollidone, polyvinyl alcohol, polyethylene glycol, methyl cellulose, hydroxymethyl cellulose, hydroxyethyl cellulose, carboxymethyl cellulose, carboxymethyl chitosan, poly-2-hydroxyethyl-meth-acrylate, polylactic acid, polyglycolic acid, collagen, gelatin plastics, and extracellular matrix proteins and their derivatives. By placing a solution or suspension in the loading well of the casting device, one can easily incorporate any combination of cells and bioactive compounds presented within the lumen of each microchannel. Of particular interest is the combination of growth factors and extracellular matrix molecules with or without cells.

In a preferred embodiment, a slow release formulation is prepared as nano- or micro-spheres in a size distribution range suitable for cell attachment and drug delivery. The spheres are embedded in the hydrogel scaffold partially exposed to the luminal surface of the multiple microchannels. The anchored intra-luminal particles function as a method for selectively restricting the delivery of cell effectors, promoters or inhibitors, and provide cellular anchoring points for cell development within the lumen of the conduits. Several molecules, pharmacological agents, neurotransmitters, genes, or other agents may be entrapped in the biodegradable polymer-manufactured micro- or nano-spheres for on-demand drug and gene delivery within the microchannels. Systems may be tailored to deliver a specified factor for cell attachment and growth, such as acidic and basic fibroblast growth factors, insulin-like growth factors, epidermal growth factors, bone morphogenetic proteins, nerve growth factors, neurotrophic factors, TGF-b, platelet derived growth factors, or vascular endothelial cell growth factor, as well as active fragments or analogs of any of the active molecules.

The disclosed devices are also amenable for controlling the loading and subsequent maintenance dose of these factors by manipulating the concentration and percentage of molecular incorporation in the micro- or nano-sphere, and the shape or formulation of the biodegradable matrix. In certain embodiments of the invention, the controlled release material includes an artificial lipid vesicle, or liposome. The use of liposomes as drug and gene delivery systems is well known to those skilled in the art. Further, the present disclosure provides for pharmaceutically acceptable delivery of neural molecules such as neuroactive steroids, neurotransmitters and their receptors. Yet another aspect of the disclosure is the manipulation of factors that modulate or measure the ionic transport across cell membranes.

Suitable biodegradable polymers can be utilized as the controlled release material. The polymeric material may be a polylactide, a polyglycolide, a poly(lactide-co-glycolide), a polyanhydride, a polyorthoester, polycaprolactones, polyphosphazenes, polysaccharides, proteinaceous polymers, soluble derivatives of polysaccharides, soluble derivatives of proteinaceous polymers, polypeptides, polyesters, and polyorthoesters or mixtures or blends of any of these. The polysaccharides may be poly-1,4-glucans, e.g., starch glycogen, amylose, amylopectin, and mixtures thereof. The biodegradable hydrophilic or hydrophobic polymer may be a water-soluble derivative of a poly-1,4-glucan, including hydrolyzed amylopectin, hydroxyalkyl derivatives of hydrolyzed amylopectin such as hydroxyethyl starch (HES), hydroxyethyl amylose, dialdehyde starch, and the like. Other useful polymers include protein polymers such as gelatin and fibrin and polysaccharides such as hyaluronic acid. It is preferred that the biodegradable controlled release material degrade in vivo over a period of less than a year. The controlled release material should preferably degrade by hydrolysis, and most preferably by surface erosion, rather than by bulk erosion, so that release is not only sustained but also provides desirable release rates. The disclosure also provides for the use of the micro-structures or nano-domains as a means to evaluate cellular function either through a calorimetric or calorimetric molecular or physiological indicator.

The present disclosure is not limited to regeneration of nerve cell connections or to nerve tissue of either the central or peripheral nerve systems. While specific alternatives to steps of the invention have been described herein, additional alternatives not specifically disclosed, but known within the art, are intended to fall within the scope of the present inventions. Thus it is understood that other applications of the present disclosure will be apparent to those skilled in the art upon the reading of the described embodiments and a consideration of the claims and drawings.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

Sciatic Nerve Repair

Preclinical data on animal models was obtained to evaluate surgical morbidity, immunogenicity, and cellularity of the implants. Using the sciatic nerve gap repair model, two separate cohorts of rats repaired with either seven or fourteen multi-luminal BNIs were examined and compared to animals repaired with empty tubes, tubes filled with collagen, or autologous grafts. Some of the animals were implanted with PTFE Micro-Renathane® tubing that included conical perforations.

As expected, the recovered implant showed a nerve cable 10 weeks after implantation (FIG. 4). The benefit of the perforations to the polyurethane Micro-Renathane® tubing is also illustrated in FIG. 4. In sharp contrast to the single nerve cable that characterizes the autograft (FIG. 4A) and the simple tubularization repair method (FIG. 4B), multiluminal repair revealed fascicular-like nerve growth throughout the length of the multiluminal BNIs 10-16 weeks after injury (FIG. 4C-H). In most cases, the nerve cables were similar in thickness and occupied all the available area within each microchannel (approximately 250 μm ID) of BNIs that contained 7 or 14 channels. Vascularization of the BNI nerve cables was observed both inside each microchannel (FIG. 4E), and along the mesenchimal layer that formed between the inside tubing and the outer agarose (FIG. 4F). No gross evidence of inflammation or tissue reaction was observed in any of the BNI-implanted animals.

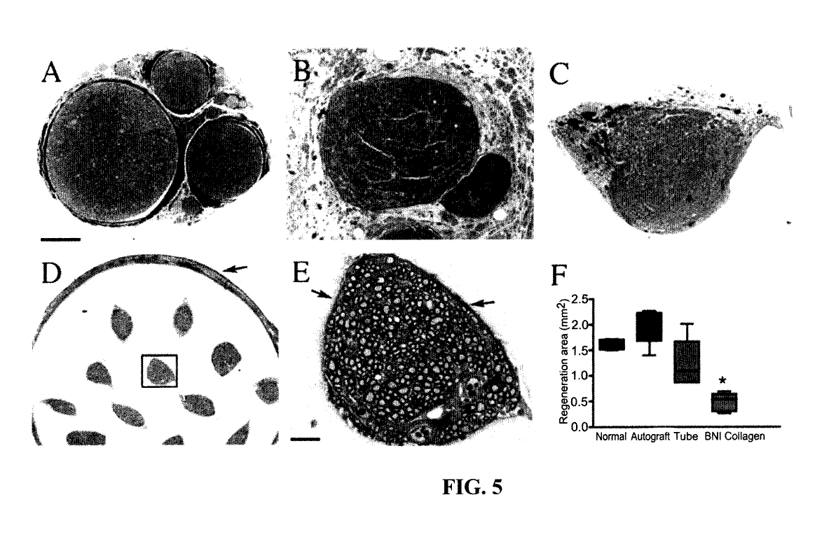

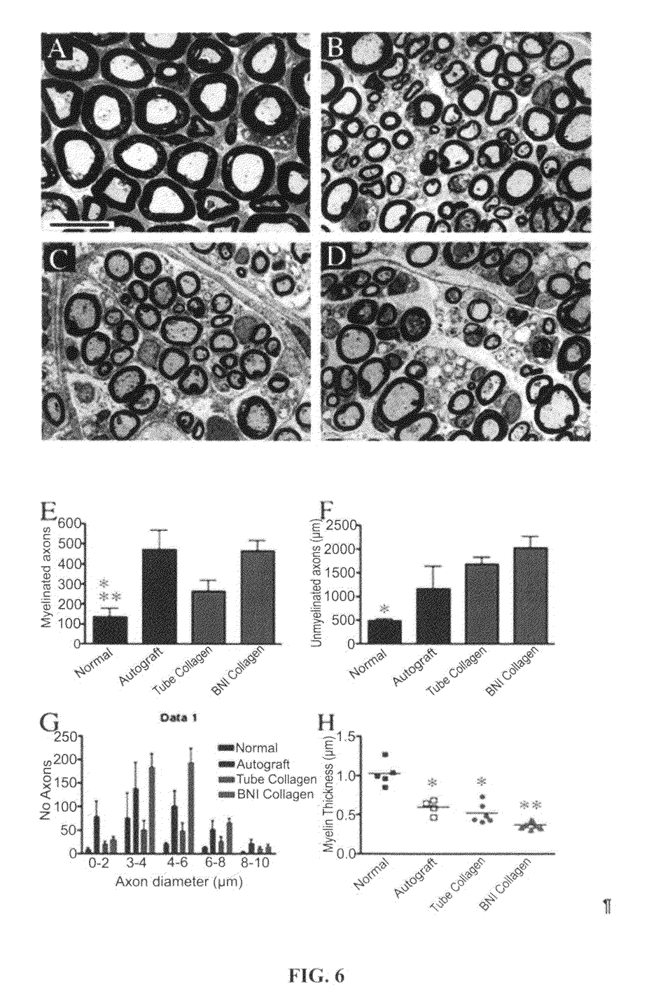

To confirm that the gross tissue regeneration observed within the BNI was filled with nerve-associated cellular structures we performed histological and morphometric analysis., as shown in FIG. 5. Compared to the uninjured controls (FIG. 5A), autograft repair (FIG. 5B) or collagen-filled tubing repairs (FIG. 5C), qualitative normal nerve regeneration was facilitated by the BNI (FIG. 5D, E). A highly vascularized mesenchimal layer covered the outer surface of the BNI hydrogel (FIG. 5D). Furthermore, each channel was vascularized and filled with numerous axons, and was surrounded by a perineurium-like outer membrane that resembled the multifascicular architecture of the normal nerves (FIG. 5E).

We then evaluated whether the total area occupied by the regenerative axons differed among the repair methods. The total area of tissue regeneration was determined by tracing the area of toludine-blue stained tissue containing visible nerve growth. The area occupied by the regenerated nerves was comparable among the autograft-repaired and collagen-loaded tabularized animals, and was similar to nerves of uninjured animals (FIG. 5F). In contrast, a three-fold reduction in the regenerated area was observed in animals repaired with the 14-channel BNI (FIG. 5F).

To determine the efficacy of nerve growth in the BNI-repaired animals, we evaluated the tissue using electron microscopy and performed morphometric analysis as shown in FIG. 6. As expected, compared to the uninjured controls, injured animals in all groups showed a qualitative increase in axon density and reduction in myelin thickness (FIG. 6A). Myelinated and unmyelinated axons in the BNI (FIG. 6D) were comparable to those in the autograft and the collagen-filled tube repairs (FIG. 6B,C). Quantification of the number of axons per fixed area (0.033 mm²; see Methods) revealed a significant increase in the density of both myelinated and unmyelinated axons in all the injured groups, compared to the uninjured controls (FIG. 6E,F). The apparent sprouting of myelinated axons was more pronounced in the autograft and BNI groups, compared to the tube/collagen repairs (5-fold and 3-fold, respectively, compared to uninjured controls). Axonal sprouting of unmyelinated axons was also evident. A 3-4 fold increase was documented in all repair groups compared to the uninjured control, with the highest number present in the BNI group (FIG. 6F). The increased number of axons in the autograft and the BNI, together with the significant reduction in the total area available for regenerative growth in the 14-channel BNI, indicated that axonal density within the BNI was increased four-fold compared to that in the autograft.

To evaluate whether specific neuron subtypes are preferentially influenced by the different repair strategies, we studied the distribution axon diameters in the regenerated nerves (FIG. 6G). The number of axons (per standardized area) was lowest in the uninjured control for all axon groups except in the 2-4 μm range, where axon number was increased over that in the collagen-loaded tube-repaired group. Both the autograft and the BNI groups demonstrated the highest increase in axonal number for all diameter ranges. However, small-diameter axons (<4 μm) were more abundant in the autograft group, whereas axons at the 2-6 μm diameter range were more prevalent within the BNI. To evaluate the “maturity” of the regenerative process, we measured the myelin thickness in all groups. As expected, myelination was thicker in the uninjured controls, and significantly reduced in all other groups. However, the axons in the autograft and tube collagen groups showed increased myelin thickness compared to the BNI (FIG. 6H).

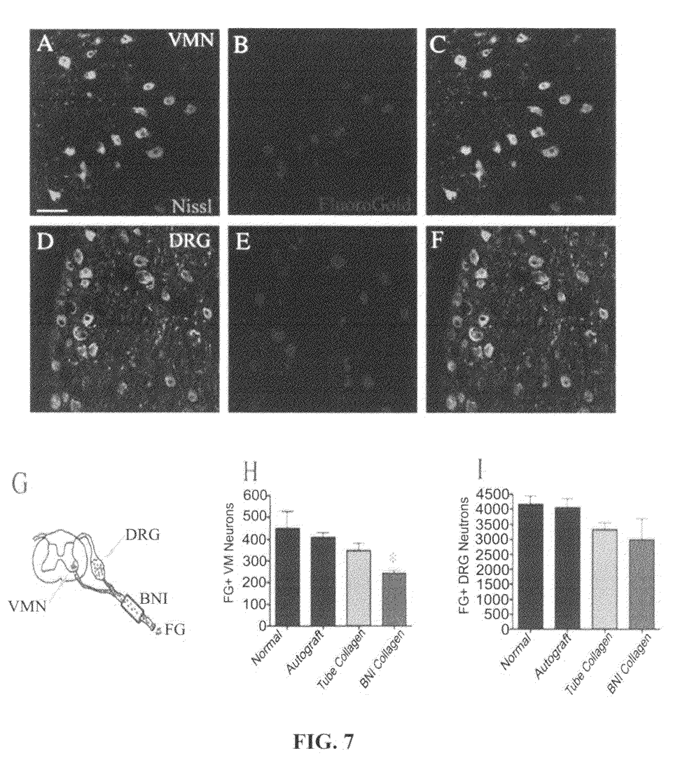

A separate group of animals underwent Fluoro-Gold (FG) tract-tracing of the sciatic nerve distal to the graft, as shown in FIG. 7. Numerous FG+ cells were visualized in the ventral motor neuron pool of the spinal cord (VMN; FIG. 7A-C) and in the sensory dorsal root ganglia (DRG; FIG. 7D-F) in Nissl counter-stained sections, as expected from their anatomical contribution to the sciatic nerve (FIG. 7G). The number of FG+ motorneurons in the BNI-repair animals (20-40% reduction) was significantly less when compared to the uninjured, autograft and tube/collagen groups (FIG. 7H). Conversely, the number of FG+ sensory neurons was statistically comparable among all the groups, with a trend for reduced FG+ neurons in both the tube/collagen and the BNI-repaired animals (FIG. 71). This data indicates that both sensory and motor axons spontaneously regenerate in all repair strategies.

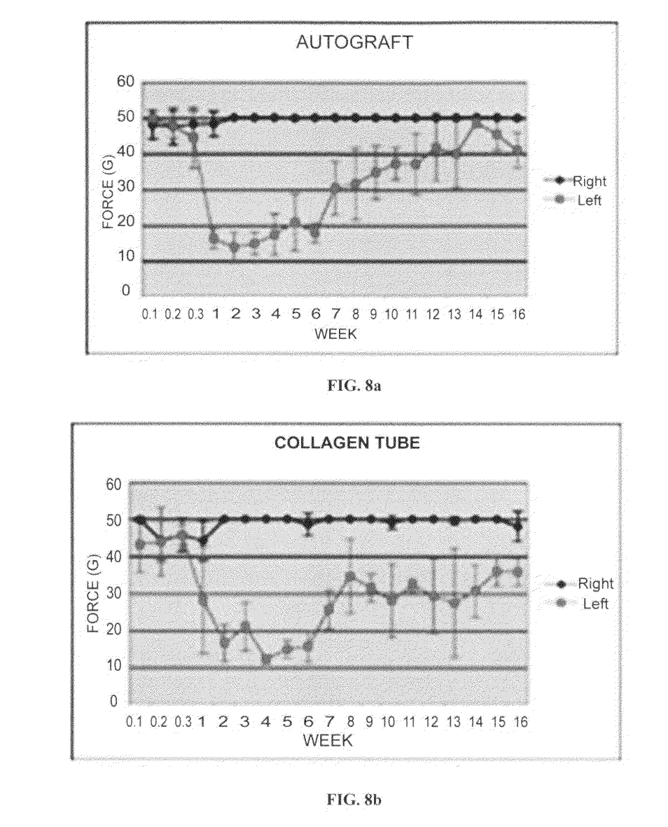

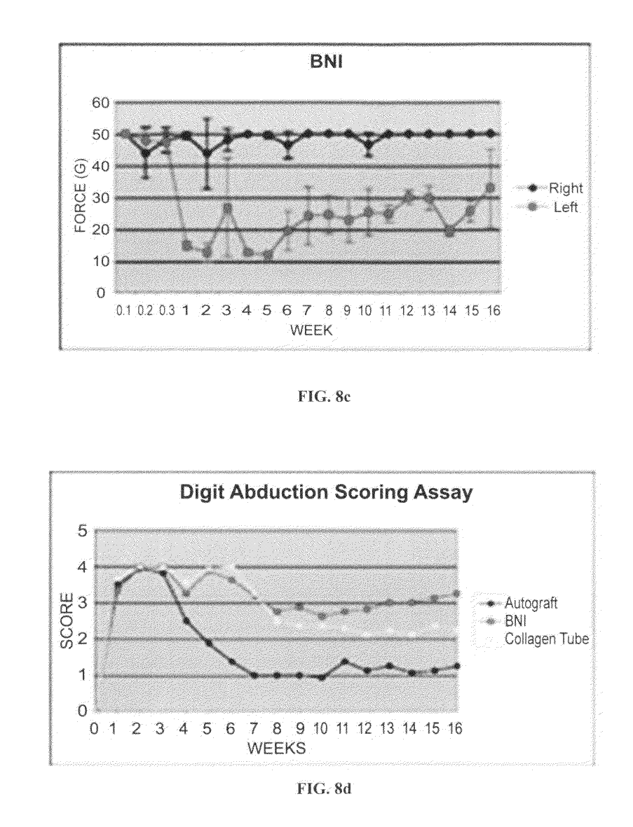

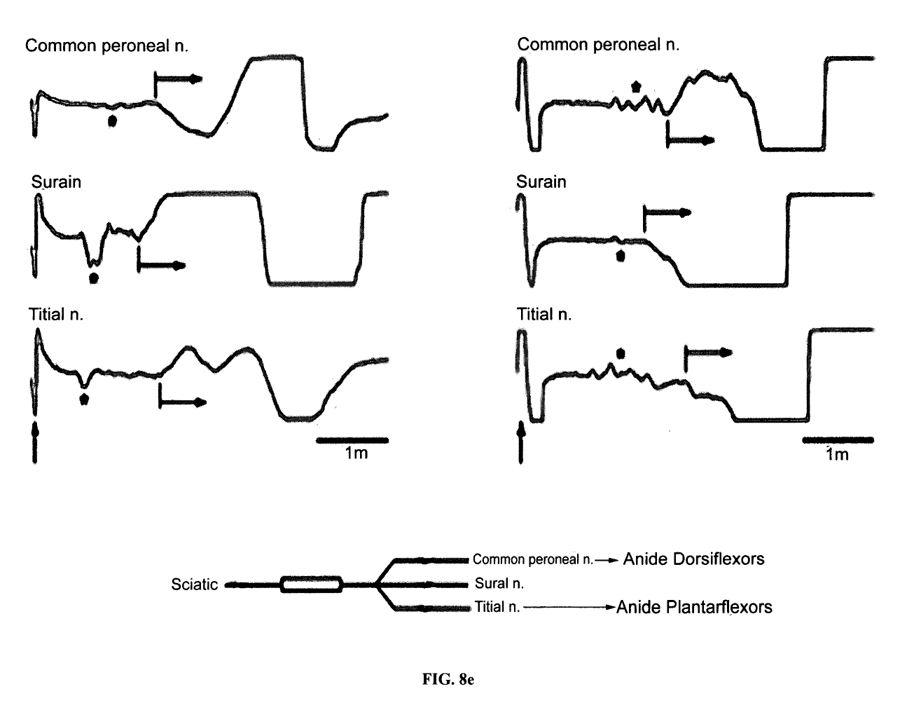

The behavioral recovery of the rats was evaluated by the dynamic plantar aesthesiometer test (FIG. 8A-C) and the digit abduction assay (FIG. 8D). As expected, the normal response of the hindlimb to mechanical stimulation (50 g) of the plantar surface declined after injury, reflecting the lack of force opposition caused by muscle denervation. The required force to elicit a response increased progressively over 16 weeks. Such recovery reached comparable levels to baseline and to the contralateral control limb in the autograft group (FIG. 8A), and progressed, albeit less effectively, in the tube/collagen and BNI repaired animals (FIG. 8B,C). These data suggest that the functional sensory regeneration of the paw plantar surface in the tube/collagen and the BNI groups remains suboptimal compared to that obtained with the autograft repair method. The Digit Abduction Score (DAS) was used to evaluate motor neuron functional regeneration (Aoki KR, 1999; FIG. 8D). Baseline measurements were normal for all treatment groups and significantly increased to the worst score (4) immediately after injury to the sciatic nerve. The recovery of animals with autografts was noted as early as 4 weeks post injury (p.i.) and reached their best score (1) at 7 weeks p.i. Conversely, those repaired with either a tube/collagen or BNIs, reached their best score (2.5) at 8 weeks p.i., with slight improvement to score of (3) at week 12 in the collagen/tube group (FIG. 8D).

We tested the electrical conduction of the regenerated nerve by stimulating the proximal end of the sciatic nerve, and recording in the common peroneal, sural and tibial branches of the sciatic nerve distal to the implant (FIG. 8E). In the simple tubularization repair, a single compound action potential was recorded in the sural and tibial nerves, but not in the peroneal nerves (FIG. 8F). In contrast, recordings from the BNI showed multiple compound potentials, which were detected in the common peroneal tibial and sural nerves (FIG. 8G). Large myoelectric depolarizations were observed in all cases, indicating the capacity of the regenerated nerves to elicit muscle contraction.

Central Nervous System Injury Repair

The tissues into which the BNI may be introduced to induce nervous tissue regeneration include those associated with neurodegenerative disease or damaged neurons. Non-limiting examples of neurodegenerative diseases which may be treated using the methods described herein are Alzheimer's disease, Pick's disease, Huntington's disease, Parkinson's disease, cerebral palsy, amyotrophic lateral sclerosis, muscular dystrophy, multiple sclerosis, myasthenia gravis, and Binswanger's disease.

Injury to the adult mammalian spinal cord results in extensive axonal degeneration, variable amounts of neuronal loss, and often-severe functional deficits. Restoration of controlled function depends on regeneration of these axons through an injury site and the formation of functional synaptic connections. Resorbable PLA tubing has been studied as a possibility to bridge the injured spinal cord (Oudega, et al. Biomaterials 22, 1125-36, 2001). Clearly, the BNI design can be adapted for spinal cord repair.

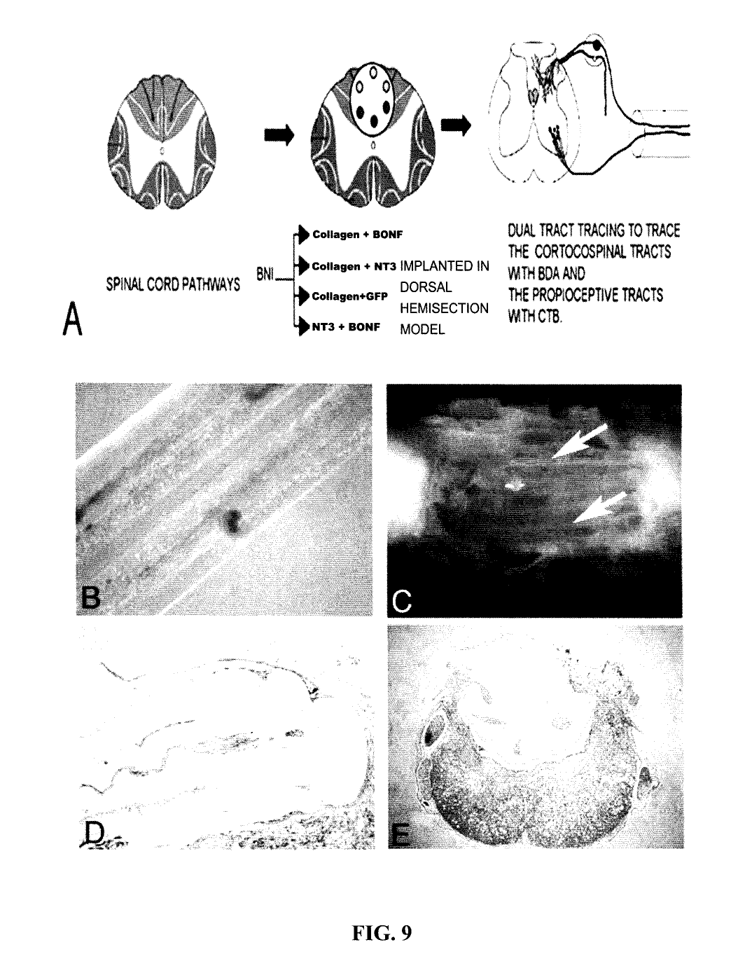

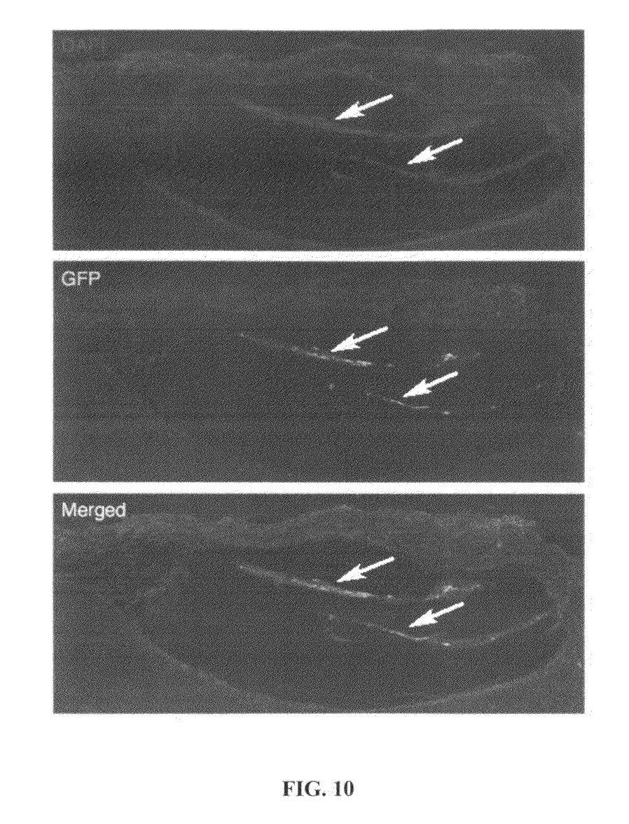

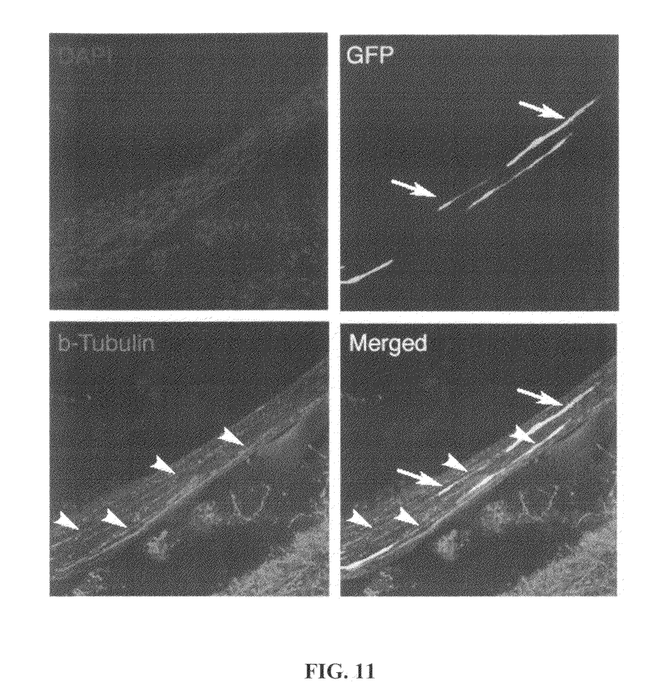

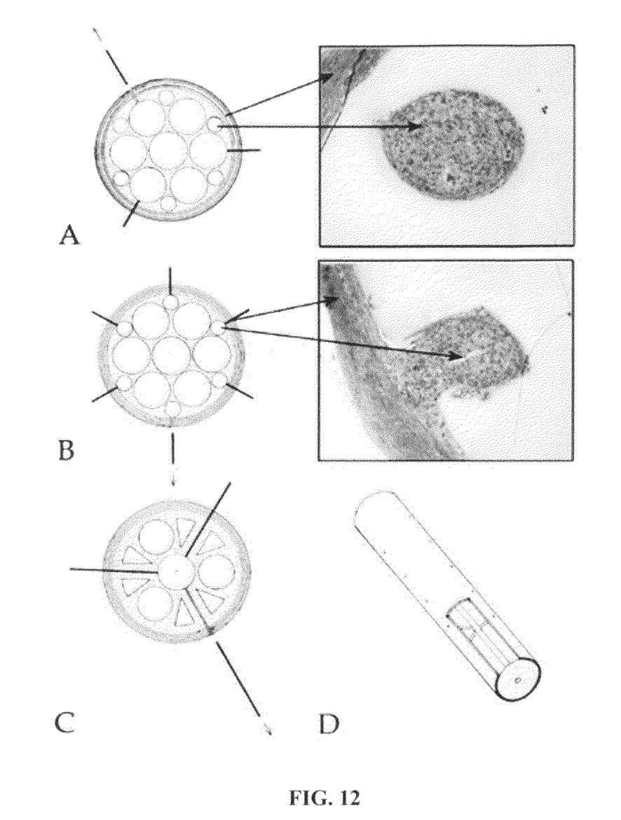

We implanted animals that underwent dorsal hemisection injury of the spinal cord with BNIs that contained channels filled with collagen only, or with collagen mixed with Schwann cells that expressed the reporter green fluorescent protein (GFP). FIG. 9 demonstrates the use of the BNI implant in repairing the injured spinal cord, as shown in photographs of the injured spinal cord 45 days after repair, which show regenerated tissue inside each microchannel (arrows). Photograph of the injured spinal cord 45 days after repair visualized by the nuclear staining DAPI demonstrates numerous cells filling the BNI microchannels. The implanted GFP-labeled Schwann cells survived inside the microchannels as indicated in the GFP and Merged photographic panels in FIG. 10. FIG. 11 shows a higher magnification of the regenerated tissue inside a BNI microchannel in the injured spinal cord, 45 days after repair. Numerous cells are visualized inside the microchannel as indicated by the nuclear staining DAPI. The implanted GFP-labeled Schwann cells survived inside the microchannels as indicated in the GFP, and more importantly numerous regenerated axons are visualized with the specific neuronal marker b-tubulin (arrow heads in FIG. 11). Thus, demonstrating the successful nerve regeneration in the adult injured spinal cord though BNI bridge repair.

In addition, damaged neurons caused by vascular lesions of the brain and spinal cord, trauma to the brain and spinal cord, cerebral hemorrhage, intracranial aneurysms, hypertensive encephalopathy, subarachnoid hemorrhage or developmental disorders may be treated using the methods provided by the present disclosure. Examples of developmental disorders include, but are not limited to, a defect of the brain, such as congenital hydrocephalus, or a defect of the spinal cord, such as spina bifida.

Non-limiting examples of tissues into which the BNI method may be used to foster and induce regeneration include fibrous, vesicular, cardiac, cerebrovascular, muscular, vascular, transplanted, and wounded tissues. Transplanted tissues are for example, heart, kidney, lung, liver and ocular tissues. In further embodiments of the invention the BNI design is used to enhance wound healing, organ regeneration and organ transplantation, including the transplantation of artificial organs.

### Materials and Methods

Hydrogel Scaffold Preparation and Cellular Loading

Agarose, a natural polymer widely used as a biomaterial for tissue engineering with demonstrated safety and biocompatibility, was experimentally selected as matrix. Multiple plastic fibers (0.25×17 mm) were placed inside the custom-made casting device. Ultrapure agarose was dissolved in sterile 1×PBS, injected into a perforated Micro-Renathane® tubing (Braintree Scientific, Inc; OD 3 mm, ID 1.68 mm, and length of 12 mm) previously placed into the casting device, and with various plastic fibers (i.e. 7 or 14) running longitudinally through the tube for channel casting and polymerized at room temperature for 15 minutes.

Cell Culture and Cell Loading

Syngenic cultures of Schwann cells were obtained from adult rat sciatic nerves and expanded in vitro according to established methods (Mathon et al., Science 291, 872-5, 2001). In order to enhance cellular attachment and growth, the cells are mixed with 10% matrigel or collagen-I prior to seeding. The cell suspension is then added to the loading chamber of the casting device and by carefully removing the fibers, the cells are drawn into the microchannels of the agarose matrix by negative pressure. The cellular density inside the channels can be varied through the use of different cell titers at the time of seeding.

The conduits are then seeded with several types of cells. In the preferred embodiment Schwann cells obtained from rodent sciatic nerves culture in DMEM/10% FBS, supplemented with forskolin, pituitary gland extract and herregulin, were seeded within the microchannels by placing the cell suspension into the loading well and then removing the synthetic fibers (FIG. 1, panel B (c-d)). By this method, both the channel casting and cellular loading can be done within minutes, in a simple and reproducible manner.

Surgery

Under anesthesia induced subcutaneously (Ketamine 87 mg/kg/Medetomidine 13 mg/kg), the left sciatic nerve was exposed through a dorsolateral incision of the gluteal muscles. A 5-7-mm segment was then excised proximal to the bifurcation of the sciatic nerve. In animals receiving an autograft, the excised segment of the sciatic nerve was immediately sutured back. Those in the tube and BNI groups were repaired using 10-0 sutures to co-apt the nerve stumps with the Micro-Renathane® tubing. The muscle was sutured and overlying skin clipped. Post-operatively the animals received Atipamezole 1 mg/kg, and were allowed to recover for 16 weeks.

Behavioral Testing

The animals were tested for recovery of motor and sensory function. Sensation was evaluated using the dynamic plantar aesthesiometer test (Ugo Basile). After a 5 min habituation period, a metal filament applied increasing pressure to the plantar surface until the rat withdrew the paw. The actual force at which the paw was withdrawn was recorded from both the injured and contralateral paws. The Digit Abduction Score (DAS) assay semiqualitatively measures muscle weakness (Aoki KR, 1999), and was used to evaluate motor axon reinnervation. Briefly, the animals were tail-suspended to elicit hindlimb extension and digit abduction. The extended hind limbs were photographed each week and digit abduction scored on a five-point scale (0=normal to 4=maximal reduction in digit abduction and leg extension) by two observers blind to the treatment.

Retrograde Tracing

A subset of animals (n=4 per group) was evaluated for anatomical regeneration using a fluorescent retrograde tract-tracer from the sciatic nerve distal to the implant. FluoroGold (FG: Fluorochrome, Englewook Colo., USA) crystals were placed for 10 min on the regenerated nerve transected distal to the repaired site. The nerve stump was then carefully rinsed, the skin sutured, and the animals given Atipamezole (1 mg/kg) during the recovery period. The animals were allowed to survive for six days prior to tissue harvesting. FG-positive cells with clear nuclei were counted in a subset of sections obtained from the dorsal root ganglion and the ventral horn of the spinal cord.

Immunostaining

Cells or tissues were incubated with a combination of primary antibodies against acetylated b-tubulin (1:200; Sigma) and S-100 (1:500 Sigma) to identify axons and Schwann cells, respectively. Visualization was achieved by tissue incubation in Cy2- and Cy3-conjugated secondary antibodies (1:400; Jackson Labs, West Grove, Pa.). Neurotrace (1:250: Molecular Probes.) was used as fluorescent Nissl conterstain. The staining was evaluated using a Zeiss Pascal confocal microscope.

Electron Microscopy and Histomorphometry

Animals were euthanized with pentobarbital and perfused with PBS followed by 4% paraformaldehyde. Overnight post-fixation was done by placing the tissue in 2% glutaraldehyde/1% paraformaldehyde/0.15M sodium cacodylate, pH 7.2 at 4° C. Tissues were rinsed, stained in 2% uranyl acetate, dehydrated, and infused in propylene oxide/Durcupan (Fluka Chemika-BioChemika, Ronkonkoma, N.Y.), in 25/75 ratio, for 1 hr at room temperature. Sciatic nerves were flat embedded in fresh Durcupan resin and polymerized 24-36 hours at 65° C. One gm thick sections were stained in Toluidine blue. Thin sections were viewed at 60 kv and photographed on a JEOL 100 CX conventional transmission electron microscope. For quantification, twenty-one pictures were taken of each nerve cross-section at random covering 1575 μm² per picture, and totaling 0.033 mm² in sampling area per animal. A MACRO (Zeiss, Co.) was written to evaluate the number of myelinated axons, axon diameter and myelin thickness in each electron micrograph, which was validated by direct comparison with measurements obtained manually. The number of unmyelinated axons was estimated manually from photographic prints. Raw data was analyzed by ANOVA followed by Neuman-Keuls multiple comparison post hoc test (Prism 4; GraphPad Software Inc.).

Modification of the Multi-Channel Luminal Surface

Synthetic or metal fibers measuring 250 micrometers in diameter by 18 millimeters in length were dipped in matrigel (ECM) forming a five-micrometer film coating. The ECM coated fibers were allowed to polymerize at room temperature for ten minutes and then rolled across a monolayer of 10 micrometer latex beads. In this manner, the beads were partially embedded into the ECM coating of the fibers. The ECM coated, bead embedded fibers were inserted into a multi-channel matrix casting device. Next, 1.5% ultrapure agarose, 1× phosphate buffered saline solution was heated to its boiling point and poured into the casting well. The agarose was allowed to polymerize at room temperature. It is contemplated that in cases in which various degrees of gel opacity are desired, various gelling agents are used with the present disclosure, including, but not limited to chitosan, collagen, fibrinogen, and other hydrogels. The beads embedded in the ECM are partially embedded and have an exposed surface. When liquid agarose is poured into the casting well, this exposed bead surface becomes embedded into the agarose matrix. Since ECM is a hydrophilic gel substance and agarose is a hydrogel matrix, when the fiber is extracted, the ECM embedded beads are released from their attachment points on the fiber and remain anchored in the luminal wall of the resulting conduit, presenting a bead surface area that is now exposed to the lumen of the conduit.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Al-Majed, A. A., Neumann, C. M., Brushart, T. M., and Gordon, T. (2000). Brief electrical stimulation promotes the speed and accuracy of motor axonal regeneration. J Neurosci 20, 2602-2608.
Boyd, J. G., and Gordon, T. (2003). Glial cell line-derived neurotrophic factor and brain-derived neurotrophic factor sustain the axonal regeneration of chronically axotomized motoneurons in vivo. Exp Neurol 183, 610-619.
Dahlin, L. B., and Lundborg, G. (2001). Use of tubes in peripheral nerve repair. Neurosurg Clin N Am 12, 341-352.
Evans, G. R. (2001). Peripheral nerve injury: a review and approach to tissue engineered constructs. Anat Rec 263, 396-404.
Franz, C. K., Rutishauser, U., and Rafuse, V. F. (2005). Polysialylated neural cell adhesion molecule is necessary for selective targeting of regenerating motor neurons. J Neurosci 25, 2081-2091.
Gordon, T., Sulaiman, O., and Boyd, J. G. (2003). Experimental strategies to promote functional recovery after peripheral nerve injuries. J Peripher Nerv Syst 8, 236-250.
Hadlock, T., Sundback, C., Hunter, D., Cheney, M., and Vacanti, J. P. (2000). A polymer foam conduit seeded with Schwann cells promotes guided peripheral nerve regeneration. Tissue Eng 6, 119-127.
Kline, D. G., Kim, D., Midha, R., Harsh, C., and Tiel, R. (1998). Management and results of sciatic nerve injuries: a 24-year experience. J Neurosurg 89, 13-23.
Lundborg, G. (2000). A 25-year perspective of peripheral nerve surgery: evolving neuroscientific concepts and clinical significance. J Hand Surg [Am] 25, 391-414.
Madison, R. D., Archibald, S. J., Lacin, R., and Krarup, C. (1999). Factors contributing to preferential motor reinnervation in the primate peripheral nervous system. J Neurosci 19, 11007-11016.
Moore, M. J., Friedman, J. A., Lewellyn, E. B., Mantila, S. M., Krych, A. J., Ameenuddin, S., Knight, A. M., Lu, L., Currier, B. L., Spinner, R. J., et al. (2006).
Multiple-channel scaffolds to promote spinal cord axon regeneration. Biomaterials 27, 419-429.
Ngo, T. T., Waggoner, P. J., Romero, A. A., Nelson, K. D., Eberhart, R. C., and Smith, G. M. (2003). Poly(L-Lactide) microfilaments enhance peripheral nerve regeneration across extended nerve lesions. J Neurosci Res 72, 227-238.
Nilsson, A., Dahlin, L., Lundborg, G., and Kanje, M. (2005). Graft repair of a peripheral nerve without the sacrifice of a healthy donor nerve by the use of acutely dissociated autologous Schwann cells. Scand J Plast Reconstr Surg Hand Surg 39, 1-6.
Schmidt, C. E., and Leach, J. B. (2003). Neural tissue engineering: strategies for repair and regeneration. Annu Rev Biomed Eng 5, 293-347.
Stokols, S., and Tuszynski, M. H. (2006). Freeze-dried agarose scaffolds with uniaxial channels stimulate and guide linear axonal growth following spinal cord injury. Biomaterials 27, 443-451.
Weber, R. A., Breidenbach, W. C., Brown, R. E., Jabaley, M. E., and Mass, D. P. (2000). A randomized prospective study of polyglycolic acid conduits for digital nerve reconstruction in humans. Plast Reconstr Surg 106, 1036-1045; discussion 1046-1038.
Witzel, C., Rohde, C., and Brushart, T. M. (2005). Pathway sampling by regenerating peripheral axons. J Comp Neurol 485, 183-190.
Yang, Y., De Laporte, L., Rives, C. B., Jang, J. H., Lin, W. C., Shull, K. R., and Shea, L. D. (2005). Neurotrophin releasing single and multiple lumen nerve conduits. J Control Release 104, 433-446.
Yoshii, S., Oka, M., Shima, M., Taniguchi, A., and Akagi, M. (2003). Bridging a 30-mm nerve defect using collagen filaments. J Biomed Mater Res A 67, 467-474.

What is claimed is:

1-35. (canceled)

36. A casting device for production of a nerve growth conduit, the casting device comprising:
a matrix casting tube;
a matrix casting tube protective shield comprising a male coupling portion joinable to a female coupling portion, wherein the joined portions encase the matrix casting tube;
microchannel forming fibers;
a fixing point for holding one end of the microchannel forming fibers;
loading fiber guideholes for placement of the microchannel forming fibers;
one or more ports for injection of matrix material into the casting tube; and
a cell suspension loading well in fluid communication with the matrix casting tube when the device is fully assembled.

37. The device of claim 36 wherein the casting device comprises a coupling ring configured to couple the matrix casting tube protective shield to the cell suspension loading well, and wherein the coupling ring further comprises a guide for the microchannel forming fibers in fluid communication with the cell suspension loading well.

38. The device of claim 36 further comprising a biopolymer injection overflow port.

39. The device of claim 36 further comprising an internal cell-suspension loading well air bleeder port.

40-45. (canceled)

46. The device of claim 36, wherein the cylindrical microchannel forming fibers have a diameter of from 50 to 500 μm.

47. The device of claim 36, wherein the microchannel forming fibers are coated with micro-structures or nano-domains.

48. The device of claim 47, wherein the micro-structures are beads.

49. The device of claim 36, wherein the matrix casting tube protective shield is sized to contain a matrix casting tube external conduit with a diameter of from 1.7 mm to 11 mm, and a length of from 0.3 cm to 30 cm.
